(19) United States
(12) Patent Application Publication
(10) Pub. No.: US 2003/0175914 A1
(43) Pub. Date: Sep. 18, 2003

(54) Method for producing glycerides of conjugated, polyunsaturated fatty acids on the basis of their alkyl esters

(76) Inventors: Kai-Uwe Baldenius, Ludwigshafen (DE); Arne Ptock, Ludwigshafen (DE)

Correspondence Address: NIXON & VANDERHYE, PC, 1100 N GLEBE ROAD, 8TH FLOOR, ARLINGTON, VA, 22201-4714, US

(21) Appl. No.: 10/380,180

(22) PCT Filed: Sep. 19, 2001
(86) PCT No.: PCT/EP01/10806

(30) Foreign Application Priority Data
Sep. 20, 2000 (DE) 100 46 879.9

(51) Int. Cl. C12P 7/64

(52) U.S. Cl. 435/134

(57) ABSTRACT

The invention relates to a method for producing glycerides that contain conjugated, polyunsaturated fatty acids by reacting the alkyl ester of the conjugated polyunsaturated fatty acids with glycerol or glycerides and concurrent lipase catalysis.

The invention relates to methods for the lipase-catalyzed preparation of glycerides comprising conjugated polyunsaturated fatty acids, preferably triglycerides, from the corresponding alkyl esters of the conjugated polyunsaturated fatty acids and glycerol or glycerides. Particular preference is given to the method for alkyl esters of conjugated linoleic acids (CLA). The use of positionally nonselective lipases from microorganisms of the genera Burkholderia, Pseudomonas, Candida, Geotrichum, Chromobacterium and Aspergillus is preferred.

Compared to other polyunsaturated fatty acids, conjugated polyunsaturated fatty acids are rather rare. Examples of conjugated fatty acids are the conjugated linoleic acids (CLA), α-parinaric acid (18:4 octadecatetraenoic acid), eleostearic acid (18:3 octadecatrienoic acid), the conjugated linolenic acids, dimorphecolic acid and calendulic acid (see diagram 1).

Diagram 1: Conjugated polyunsaturated fatty acids

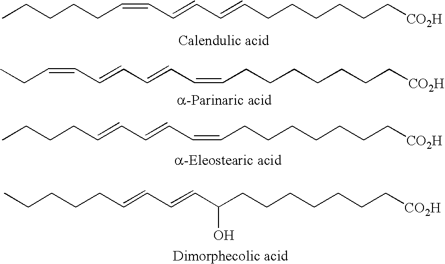

CLA is a collective term for positional and structural isomers of linoleic acid, which are distinguished by a conjugated double bond system starting at carbon atom 8, 9, 10 or 11. A few examples are depicted in diagram 2.

Geometric isomers exist for each of said positional isomers, i.e. cis-cis, trans-cis, cis-trans, trans-trans.

Especially C18:2 cis-9, trans-11 and C18:2 trans-10, cis-12 CLAs which represent the biologically most active isomers, are particularly interesting, since they have proved to be cancer-preventive in animal experiments, have an anti-arteriosclerotic effect and reduce the proportion of body fat in humans and animals.

Diagram 2: Four isomers of conjugated linoleic acids

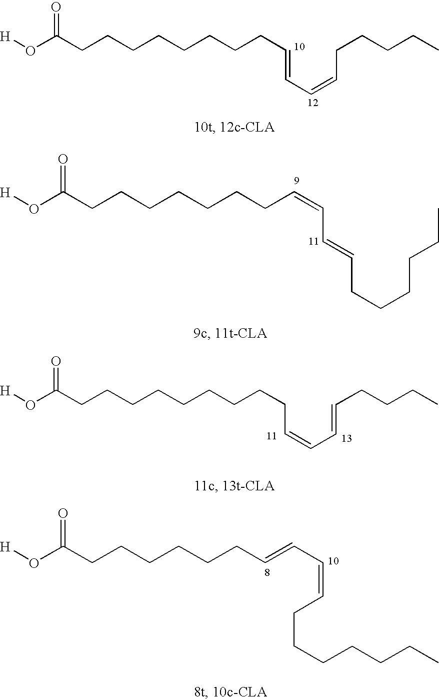

Commercially, CLAs are sold these days mainly as free fatty acids. Free fatty acids often have disadvantageous sensory properties. Triglycerides are to be preferred over free fatty acids for incorporation in food, also for technological reasons. There have been attempts therefore, to convert industrially produced free CLAs to triglycerides.

The methods described in the prior art use transesterification methods which are catalyzed enzymatically or chemically. The chemical processes are performed at high temperatures in the presence of inorganic catalysts such as, for example, sodium or sodium methoxide. Said processes are employed, for example, in margarine production for hardening, i.e. for replacing unsaturated or polyunsaturated fatty acids by saturated fatty acids. The drastic conditions, however, cause side reactions, especially in the case of unsaturated fatty acids. Side reactions include especially cis/trans isomerizations, migration of double bonds, but also hydrogenations of double bonds or crosslinking of the unsaturated fatty acids between one another (polymerization). Trans-fatty acids and all-trans-fatty acids (but not cis/trans-conjugated linoleic acids) have unfavorable physiological properties. In addition, it has been known for many years that trans-fatty acids increase the concentration of cholesterol in the serum. Therefore, cis/trans isomerization is to be avoided in the case of unsaturated fatty acids.

Enzymatically catalyzed methods are based on the use of lipases and can be carried out under distinctly milder conditions, keeping the proportion of undesired by-products at a low level.

Owing to the conjugated double-bond system, conjugated polyunsaturated fatty acids are particularly sensitive compounds which are particularly susceptible to the side reactions described above.

Lipases are enzymes catalyzing the hydrolysis of fatty ester bonds in glycerides with liberation of fatty acids (glycerol ester hydrolases). This reaction is reversible so that the enzymes can catalyze esterification too. Lipases are found in plants, animals, bacteria and fungi. Pancreatic lipase from cattle, sheep and pigs is frequently used, but is increasingly being replaced also by microbial lipases. Lipases can roughly be divided into three categories. Firstly, lipases can act positionally nonspecifically and can cleave fatty esters regardless of their type and position in the glyceride (e.g. lipases from Corynebacterium or Candida). They may, however, also have 1,3-specificity, i.e. they preferably hydrolyze the esters in position 1 and 3 of the glyceride (for example lipases from Rhizopus and Mucor). The specificity can be more or less pronounced. Specificities for particular chain lengths or even for particular fatty acids are also possible. Geotrichum candidum lipase 1 (GLC-1) has a preference for esters of long-chain fatty acids, and in addition fatty acids having a cis-Δ9 double bond are preferred (Jensen RG et al., J Am Oil Chem Soc 1965; 42(12):1029-32; Jensen RG Lipids. 1972; 7(11):738-41). Another lipase having cis-Δ9 specificity has been isolated from Candida parapsilosis (Briand D et al., Lipids 1995; 30(8),747-754). In addition, said lipase seems to have a general preference for unsaturated, long-chain fatty acids. An overview of lipases, their specificity and use can be found in Kazlauskas RJ et al. (Kazlauskas RJ et al., Biotransformation with lipases in Biotechnology, Vol.8a, eds. Rehm HJ et al., Wiley-VCH, Weinheim, Germany).

The lipase reaction is reversible so that hydrolysis and esterification can occur in parallel. This facilitates conversion of acylglycerides by means of transesterification. Commercially of interest are especially the 1,3-specific lipases which limit the otherwise very broad product spectrum of a lipase-catalyzed transesterification. The method may be used in order to enrich acyglycerides, especially triglyceride, with particular fatty acids. The methods are usually employed for the hardening of fat.

WO-91/08677 describes a transesterification method using lipases, in which a stearic acid source (stearic acid, methyl stearate or ethyl stearate) is reacted with vegetable oils. The essential object of the method is to concentrate the saturated fatty acid stearic acid in oils and fats with the aim of modifying their properties (e.g. spreadability etc.) (enzymatic hardening of fat). The lipases used in the method are restricted. Explicitly excluded are positionally nonspecific lipases from Candida, Corynebacterium, Staphylococcus, and also lipases having a preference for unsaturated fatty acids with a Δ9 double bond. In contrast, preference is given to using 1,3-specific lipases, for example from Mucor miehei and Rhizopus delemar; particularly preferred is the lipase from Mucor miehei (Novo Lipozyme 3A). The starting material employed is oils or fats.

EP-0093602 describes a continuous process for transesterification of oils or fats with fatty acids catalyzed by a 1,3-specific lipase from Aspergillus niger, Mucor, or Rhizopus species. Preference is given here to using free unsaturated fatty acids such as myristic acid, palmitic acid and stearic acid, especially in order to modify palm oil. Here too, the preferred intention is to increase the saturated fatty acid content.

EP-0305901 describes a continuous method for the transesterification of oils or fats with fatty acids or of fatty esters using specific high-molecular-weight lipases with 1,3-specificity. The molecular weight of the lipases used in the method described is 100,000 or greater. Preference is given to lipases from the species Alcaligenes, Achromobacter or Pseudomonas.

EP 866 874 describes a process for preparing materials having an enhanced fraction of certain CLA isomers by using isomer-specific lipases.

U.S. Pat. No. 5288619 describes a method for margarine production with transesterification of natural oils, which method uses a stearic acid source (stearic acid or stearic esters of short-chain monohydric alcohols) and is catalyzed by 1,3-specific lipases. The proportion of saturated fatty acids in the glyceride is expressly increased here. The starting material employed is oils or fats.

Disadvantages of the methods described above are firstly the restriction to oils and fats as starting material, and secondly the use of 1,3-specific lipases which do not allow transesterification of the 2 position. The 2 position can be reacted only via intramolecular acyl migration, which can be achieved by increased reaction times.

Lipase-catalyzed reactions of fatty acids or of fatty esters with glycerol as substrate are known.

Thus, a reaction of free linoleic acid or free conjugated linoleic acid with glycerol to give the corresponding triglycerides, catalyzed by a lipase from Mucor miehei (Arcos JA et al., Biotechnol Bioeng. 2000; 68(5):563-70), has been described. Since the lipase in the method described is a 1,3-specific lipase, long reaction times are necessasry. Triglycerides are obtainable here only after intramolecular acyl group migration and renewed acylation, so that initially mainly 1,3-diacylglycerides are obtained. Said method makes use of free fatty acids.

Yamane et al. (Yamane T et al., Ann N Y Acad Sci. 1998; 864:171-9) describe a method for the lipase-catalyzed glycerolysis of all-Z-4,7,10,13,16,19-docosahexaenoic ethyl ester using a Pseudomonas lipase. All-Z-4,7,10,13,16,19-docosahexaenoic acid is a polyunsaturated, unconjugated fatty acid.

Haraldsson describes the preparation of modified lipids by lipase-catalyzed reaction of free acids or esters of all-Z-4,7,10,13,16,19-docosahexaenoic acid or all-Z-5,8,11,14,17-eicosapentaenoic acid with triglycerides or glycerol (Haraldsson GG in Enzymes in Lipid Modification, ed. Bornscheuer UT, Wiley-VCH, Weinheim, Germany, 2000, pages 170-189). Both fatty acids are polyunsaturated unconjugated fatty acids.

EP-0779033 describes a method for preparing CLA-containing triglycerides. According to this, linoleic acid is isomerized in a conventional process at 180° C. with NaOH in ethylene glycol to give free CLA, and the free CLA is transesterified with palm oil triglycerides using an immobilized lipase from Mucor miehei. The roduct obtained is a triglyceride containing approx. 8% in each case of the two desired CLA isomers (9c, 11t- and 10t,12c-CLA) in esterified form. In a similar manner, CLA isomer mixtures which ontained individual isomers in concentrated form were transesterified with palm oil triglycerides, and a CLA content of 30% in the triglyceride was achieved (GP McNeill et al., J. Am. Oil Chem. Soc. 76 (1999) 1265). The transesterification of butter fat with free CLA is based on a similar method in which inter alia the immobilized lipase from Candida antarctica serves as the preferred catalyst (Garcia HS et al., Biotechnol. Tech. 12 (1999) 369-373; Garcia HS et al., J Dairy Sci 2000, 83:371-377; Garcia HS et al., Biotechnology Letters 1998; 20(4):393-395). Similarly, corn oil was also modified with lipase catalysis using chemically prepared CLAs (Martinez CE et al., Food Biotechnology 1999, 13(2) :183-193).

In other, nonenzymatic methods glycerol esterification and the transesterification of natural fats and oils with CLA acids are carried out with the addition of known esterification catalysts at high temperatures (180_-240_C) (JD Mikusch; Farben, Lacke, Anstrichstoffe 4 (1950) 149-159; DE-19718245). Owing to the high temperatures required in the process, the CLA-containing triglycerides produced contain a proportion of undesired isomers (in particular 8t,10c- and 11c,13t-CLA fatty acid residues) which is unacceptable for nutritional applications.

The disadvantage of the methods described above is that the triglycerides are prepared starting from CLAs in the form of free fatty acids. The conventional preparation method for free CLA acids, in which, for example, linoleic acid-containing oils (e.g. sunflower oil, soya oil or safflower oil) are isomerized using NaOH or KOH in ethylene glycol at 180_C (Ip C et al, Cancer Res. 51 (1991) 6118-6124), requires superstoichiometric amounts of alkali (based on the fatty acids contained in the oil) and results in considerable amounts of undesired CLA isomers (in particular 8t,10c- and 11c,13t-CLA). In an improved method, isomerization of linoleic acid-containing oils with KOH in propylene glycol is carried out at 150_C. Free CLA acids are obtained which contain only a small amount of undesirable isomers (EP-839897). However, this method too requires superstoichiometric amounts of KOH and corresponding amounts of mineral acids in order to liberate the free CLA acids from the CLA soaps formed. Methods based on the use of free CLA acids are therefore economically disadvantageous.

In an economically and ecologically advantageous method, it is possible to isomerize alkyl esters of linoleic acid with catalytic amounts (0.3 to 1%) of base (potassium alcoholate), resulting in CLA alkyl esters of high purity (DE-1156788 and DE-1156789).

To date, no economical method is known to prepare CLA-containing triglycerides from said CLA alkyl esters. An important demand on the preparation method is to obtain the CLA-containing triglycerides with an undesired isomer (8t,10c and 11c,13t) content of a low level similar to that present in the CLA alkyl ester starting material. This excludes the conventional chemical transesterification methods, owing to the drastic conditions and undesired by-products connected with them.

It is an object of the present invention to prepare CLA-containing triglycerides from CLA alkyl esters.

We have found that this object is achieved by the method of the invention. It differs from the methods described in the prior art.

Particularly advantageous, compared with the chemically catalyzed transesterification methods, are the mild conditions of the method of the invention with the use of lipases which, in contrast to the customary chemical transesterifications, do not attack the sensitive conjugated double bond system of the starting compounds. This leads to high degrees of purity and a cost effective preparation without major purification steps to remove undesired by-products.

In contrast to many of the abovementioned methods, the method of the invention starts from fatty acid alkyl esters and not from the free fatty acids. Said esters are, as described above, obtainable in higher purity than the free CLAs by a particularly mild, economic method without an increased proportion of undesired isomers. Up until now, no method has been disclosed with which it is possible to convert the alkyl esters of conjugated polyunsaturated fatty acids, in particular of CLAs, into glycerides.

The methods described, as are used in the hardening of fat, have the object of increasing the proportion of saturated fatty acids in the triglyceride. For this purpose, usually 1,3-specific lipases are used in order to keep the possible variations in the transesterification at a low level. In addition, lipases selectively preferring unsaturated fatty acids are sometimes expressly unpreferred. In contrast to said methods, the object of achieving a maximum possible proportion of conjugated unsaturated fatty acids in the glyceride is achieved by the method of the invention. For this purpose, positionally nonspecific lipases or lipases having a preference for unsaturated fatty acids with a cisΔ9 or transΔ10 double bond are expressly preferred, especially if triglycerides are to be obtained as the preferred end product. If triglycerides are to be obtained as the preferred product, then the use of 1,3-specific lipases such as, for example, of the lipase from Mucor miehei is less advantageous for the reaction with glycerol (see also Examples 10 and 11). However, the 1,3-specific lipases are suitable for preparing from glycerol monoglycerides and diglycerides which may be used, for example, as emulsifiers. It is also possible to use 1,3-specific lipases in the method of the invention in order to achieve a defined introduction of the conjugated polyunsaturated fatty acids in triglycerides (fats or oils) as substrate.

In general, the method of the invention may be used for alkyl esters of conjugated polyunsaturated fatty acids. All conjugated polyunsaturated fatty acids are sensitive compounds and are prone, under drastic reaction conditions, to undesired side reactions, such as, for example, polymerizations, Diels-Alder reactions and cis/trans isomerizations.

A fatty acid means an unbranched carboxylic acid having an even number of carbon atoms and at least 16 carbon atoms, preferably from 16 to 22 carbon atoms, particularly preferably from 18 to 22 carbon atoms and very particularly preferably 18 carbon atoms.

An unsaturated fatty acid means a fatty acid having at least two double bonds.

A conjugated unsaturated fatty acid means an unsaturated fatty acid having at least two double bonds conjugated with one another.

Preference is given to the alkyl esters of conjugated polyunsaturated fatty acids such as, for example, conjugated linoleic acids (CLAs), α-parinaric acid (18:4 octadecatetraenoic acid), eleostearic acid (18:3 octadecatrienoic acid), dimorphecolic acid, conjugated linolenic acids and calendulic acid, and particular preference is given to CLA preparations containing 9cis,11trans-CLA alkyl esters and 10trans,12cis-CLA alkyl esters.

Particular preference is given to CLA preparations in which the proportion of CLAs is greater than 50% and which have, in each case, a proportion of less than 1% of the 11,13-octadecadienoic ester isomers, 8,10-octadecadienoic ester isomers and trans/trans-octadecadienoic ester isomers.

Alkyl esters of the conjugated polyunsaturated fatty acids mean esters thereof with alkanols, preferably with C₁-C₅-alkanols, such as, for example, methanol, ethanol, propanol, isopropanol, n-butanol, isobutanol, tert-butanol, or n-pentanol and its isomers (2-pentanol, 3-pentanol, 2-hydroxy-3-methylbutane. Particularly preferred are methanol and ethanol.

A conjugated fatty acid-containing glyceride means a monoglyceride, diglyceride or triglyceride in which at least one carboxylic acid belongs to the conjugated fatty acids.

The method is preferably utilized for preparing mainly triglyceride-containing glyceride preparations. The proportion of triglycerides in the glyceride preparation is preferably greater than 50%, particularly preferably greater than 90%.

A glyceride means glycerol esterified with one, two or three carboxylic acid residues.

The glyceride used in the method of the invention may comprise a synthetic or naturally occurring glyceridic oil or glyceridic fat or a derivative thereof.

A preferred substrate is synthetic glycerides containing acyl radicals having from 1 to 22 carbon atoms, preferably having 18 carbon atoms.

A preferred substrate is natural oils and fats containing acyl radicals having at least 16 carbon atoms, preferably having 16 to 22 carbon atoms, particularly preferably having 18 to 22 carbon atoms, very particularly preferably having 18 carbon atoms.

Particularly preferred in this connection are natural oils and fat having a high proportion of unsaturated fatty acids such as, for example, sunflower oil, rapeseed oil, fish oil, soya oil, palm oil, safflower oil, linseed oil, wheatgerm oil, peanut oil, cottonseed oil, corn oil, shea butter, tung oil or butterfat or derivatives thereof.

A glyceride in accordance with the method of the invention further means derivatives derived from glycerol. This includes besides the fatty acid glycerides described above also glycerophospholipids and glyceroglycolipids. Preference is given here to glycerophospholipids such as lecithin (phosphatidylcholine), cardiolipin, phosphatidylglycerol, phosphatidylserine and alkylacylglycerophospholipids such as plasmalogen.

The invention relates to a lipase-catalyzed method for preparing conjugated fatty acid-containing triglycerides from the corresponding alkyl esters of the conjugated fatty acids and from glycerol or glycerides.

Lipases here in general mean enzymes which catalyze the hydrolysis of fatty ester bonds in glycerides with liberation of fatty acids (glycerol ester hydrolases) or the reverse reaction. Advantageously, the method of the invention is carried out using positionally nonspecific lipases. Particularly preferred in this connection are positionally nonspecific lipases from microorganisms such as bacteria, fungi or yeasts. Advantageously, lipases are from microorganisms of the genera Burkholderia, Pseudomonas, Candida, Geotrichum, Chromobacterium, Corynebacterium, Staphylococcus and Aspergillus. Particularly preferred are the genera and species Burkholderia plantarii, Burkholderia cepacia, Candida antarctica, Candida rugosa, Candida cylindracea, Corynebacterium acnes, Staphylococcus aureus, Geotrichum candidum, Pseudomonas cepacia, Pseudomonas fluorescens, Aspergillus niger, Candida lipolytica, Chromobacterium viscosum.

It is further advantageous to use lipases having a specificity for fatty acids with cis-Δ9 or trans-Δ10 double bonds. Particularly preferred in this connection are lipases from Candida parapsilosis and Geotrichun candidum.

The method of the invention may use the lipase as free or bound (immobilized) enzyme. The lipase used may be employed as pure protein or as more or less purified protein or as a lipase-containing cell extract. In principle, the use of lipase-containing microorganisms or preparations derived therefrom is also possible. Particularly preferred is the use of a lipase preparation applied to a solid support. Enzymes may be bound to a multiplicity of solid supports covalently or via adsorption. Suitable solid supports are Celite, silica gel, Amberlite, support materials from diverse polymers (for example polypropylenes, polystyrenes, polyurethanes, polyacrylates) or sol gels (Kazlauskas RJ et al., Biotransformation with lipases in Biotechnology, Vol.8a, eds. Rehm HJ et al., Wiley-VCH, Weinheim, Germany).

In one embodiment of the method of the invention, alkyl esters of conjugated polyunsaturated fatty acids are transesterified with glycerol to give conjugated polyunsaturated fatty acid-containing glycerides. In the process, the corresponding alkanol is liberated from the alkyl ester.

In a general embodiment, alkyl esters of the conjugated polyunsaturated fatty acids are reacted with glycerol in a ratio of from 2 to 10 mol, particularly preferably 3 to 5 mol alkyl ester per mole of glycerol. The reaction is carried out with the addition of 0.01 to 100% by weight (based on the alkyl ester), particularly preferably 1 to 10% of a lipase, and with stirring at temperatures of from 0 to 100° C., particularly preferably 30 to 80° C. In this connection it is advantageous but not absolutely necessary to remove the liberated alkanol from the reaction mixture. Said alkanol can be removed by distillation under atmospheric pressure or in vacuo. The lipase used may be employed as more or less purified protein, as a lipase-containing cell extract or as a lipase preparation applied to a solid support.

An alternative embodiment of the method of the invention comprises the lipase-catalyzed reaction of alkyl esters of the conjugated polyunsaturated fatty acids with acylglycerides (monoglycerides, diglycerides or triglycerides or mixtures thereof), such as, for example, natural oils or fats to give conjugated polyunsaturated fatty acid-containing glycerides.

In a general embodiment, alkyl esters of the conjugated polyunsaturated fatty acids are reacted with oils such as, for example, sunflower oil, rapeseed oil, fish oil, soya oil, palm oil, safflower oil, linseed oil, wheatgerm oil, peanut oil, cottonseed oil, corn oil, milk fat or shea butter in a ratio of from 1 to 10 mol alkyl ester (particularly preferably 3 to 5 mol) per mole of acylglyceride. The reaction is carried out with the addition of from 0.01 to 100% by weight (based on the alkyl ester, particularly preferably 0.2 to 10%) of a lipase with stirring at from 0 to 100° C., particularly preferably 30° to 80° C. The lipase used may be employed as a more or less purified protein, as a lipase-containing cell extract or as a lipase preparation applied to a solid carrier. It is possible for the fatty acid alkyl esters formed as a by-product in said embodiment to be removed by distillation in vacuo at below 200° C. in a subsequent process step or in the course of a continuous process.

An advantageous embodiment results from carrying out the reaction in the presence of water. Water may be introduced into the reaction mixture via the lipase preparation (commercially available lipase preparations contain protein-bound water) or by adding it to one of the reaction components or directly.

It is advantageous in the method based on glycerol to add water in an amount no higher than the amount of glycerol present in the mixture. The amount by weight of water in the reaction mixture is preferably less than 100% of the amount by weight of glycerol, particularly preferably less than 25% and very particularly preferably less than 10%.

In the method based on glycerides, it is also possible to add relatively large amounts of water. However, here too preference is given to the amount by weight of water being less than 100% of the amount by weight of glycerides in the reaction mixture.

Of the methods described above, preference is given to those which lead to glycerides mainly consisting of triglycerides and containing conjugated fatty acids. Particularly preferred are methods leading to glyceride mixtures which contain approx. 84 to approx. 95% by weight of triglycerides, approx. 5 to 15% by weight of diglycerides and less than approx. 5% by weight of monoglycerides.

The method of the invention may be carried out in the presence of organic solvents such as, for example, ethers, such as MTB, THF, dioxane or dibutyl ether, hydrocarbons, such as toluene, xylene or alkanes, halogenated hydrocarbons such as dichloromethane or ketones and nitrites, such as acetone, acetonitrile or diethyl ketone. In this connection, the amount of the organic solvent in the reaction mixture may be from 0.1 to 20 fold of the amount of all other reaction components, preferably 0.5 to 10 fold.

### EXEMPLARY EMBODIMENTS

Preparation of CLA-containing triglycerides from CLA ethyl esters:

Example 1:

A CLA ethyl ester preparation (10 g; composition: 36% 9c,11t-CLA ethyl ester, 36% 10t,12c-CLA ethyl ester, <3% of other CLA ethyl esters), glycerol (1.1 g), Burkholderia plantarii lipase (1.0 g, on polypropylene support) were stirred at 35° C. with reduced pressure (10 mbar).

Example 2:

A CLA ethyl ester preparation (10 g; composition: 36% 9c,11t-CLA ethyl ester, 36% 10t,12c-CLA ethyl ester, <3% of other CLA ethyl esters), glycerol (1.1 g), Candida antarctica lipase (10 g, supported; “Novozym 435”) were stirred at 35° C. with reduced pressure (10 mbar).

Example 3:

A CLA ethyl ester preparation (10 g; composition: 36% 9c,11t-CLA ethyl ester, 36% 10t,12c-CLA ethyl ester, <3% of other CLA ethyl esters), glycerol (1.1 g), Candida antarctica lipase (0.5 g) were stirred at 35° C. with reduced pressure (10 mbar).

Example 4:

A CLA ethyl ester preparation (10 g; composition: 36% 9c,11t-CLA ethyl ester, 36% 10t,12c-CLA ethyl ester, <3% of other CLA ethyl esters), glycerol (1.1 g), Burkholderia cepacia lipase (0.5 g) were stirred at 35° C. with reduced pressure (10 mbar)

Example 5:

A CLA ethyl ester preparation (5 g; composition: 36% 9c,11t-CLA ethyl ester, 36% 10t,12c-CLA ethyl ester, <3% of other CLA ethyl esters), glycerol (0.55 g), Burkholderia plantarii lipase (0.5 g, on polypropylene support) were stirred at 70° C. with reduced pressure (500 mbar).

Example 6:

A CLA ethyl ester preparation (5 g; composition: 36% 9c,11t-CLA ethyl ester, 36% 10t,12c-CLA ethyl ester, <3% of other CLA ethyl esters), glycerol (0.55 g), Candida antarctica lipase (0.5 g, supported; “Novozym 435”) were stirred at 70° C. with reduced pressure (500 mbar).

Example 7:

A CLA ethyl ester preparation (5 g; composition: 36% 9c,11t-CLA ethyl ester, 36% 10t,12c-CLA ethyl ester, <3% of other CLA ethyl esters), glycerol (0.55 g), Burkholderia cepacia lipase (0.25 g) were stirred at 70° C. with reduced pressure (500 mbar).

Example 8:

A CLA ethyl ester preparation (5 g; composition: 36% 9c,11t-CLA ethyl ester, 36% 10t,12c-CLA ethyl-ester, <3% of other CLA ethyl esters), glycerol (0.55 g), toluene (5 g) and Candida antarctica lipase (0.25 g, supported; “Novozym 435”) were stirred at 70° C. with reduced pressure (500 mbar).

Example 9:

A CLA ethyl ester preparation (5 g; composition: 36% 9c,11t-CLA ethyl ester, 36% 10t,12c-CLA ethyl ester, <3% of other CLA ethyl esters), glycerol (0.55 g), dioxane (5 g) and Candida antarctica lipase (0.25 g, supported; “Novozym 435”) were stirred at 70° C. with reduced pressure (500 mbar).

The result of Examples 1 to 9: After 1, 2, 4 and 7 h, small samples were removed in each case and analyzed by thin layer chromatography. An amount increasing with time of monoglycerides, diglycerides and triglycerides was detected.

Example 10:

A CLA ethyl ester preparation (5 g; composition: 36% 9c,11t-CLA ethyl ester, 36% 10t,12c-CLA ethyl ester, <3% of other CLA ethyl esters), glycerol (0.55 g), Mucor miehei lipase (0.5 g, supported; “Lipozym IM”) were stirred at 70° C. with reduced pressure (500 mbar). After 1, 2, 4 and 6 h, small samples were removed, and thin layer chromatography detected an increasing amount of glycerides, but only traces of diglycerides substituted in the middle position and of triglycerides.

Example 11:

A CLA ethyl ester preparation (5 g; composition: 36% 9c,11t-CLA ethyl ester, 36% 10t,12c-CLA ethyl ester, <3% of other CLA ethyl esters), glycerol (0.55 g), toluene (5 g) and Mucor miehei lipase (0.5 g, supported; “Lipozym IM”) were stirred at 70° C. with reduced pressure (500 mbar). After 1, 2, 4 and 6 h, small samples were removed, and thin layer chromatography detected an increasing amount of glycerides, but only traces of diglycerides substituted in the middle position and of triglycerides.

Example 12:

A CLA ethyl ester preparation (3.8 g; composition: 48% 9c,11t-CLA ethyl ester, 48% 10t,12c-CLA ethyl ester, <3% of other CLA ethyl esters), glycerol (8 g), Candida antarctica lipase (0.8 g, supported; “Novozym 435”) were stirred at 55° C. with reduced pressure (500 mbar).

Result: Small samples were taken after each of 1, 2, 4 and 7 hours and analyzed by thin layer chromatography. An increasing amount of mono-, di- and triglycerides was detected over time. For qualitative GC analysis, the immobilized lipase was removed from the reaction mixture before the reaction mixture was silylated with BSTFA (N,O-bistrimethylsilyltrifluoroacetamide). The measurement was carried out on an Optima-Delta-6.

GC area %: glycerol 39%, 9c,11t-CLA ethyl ester 19%, 10t,12c-CLA ethyl ester 19%, monoglyceride 0.2%, diglyceride 1.1%, triglyceride 2%.

We claim:

1. A method for preparing glycerides comprising conjugated polyunsaturated fatty acids, which comprises reacting alkyl esters of the conjugated polyunsaturated fatty acids with glycerol or glycerides with lipase catalysis and removing the resulting alkyl alcohol from the reaction mixture.

2. A method as claimed in claim 1, wherein the glyceride comprising the conjugated polyunsaturated fatty acids is composed mainly of triglycerides.

3. A method as claimed in claim 1 or 2, wherein the alkyl ester is a C₁- to C₅-alkyl ester.

4. A method as claimed in claims 1 to 3, which comprises selecting the conjugated polyunsaturated fatty acids from the group comprising conjugated linoleic acids, conjugated linolenic acid, calendulic acid, α-parinaric acid, dimorphecolic acid or eleostearic acid.

5. A method as claimed in claims 1 to 4, which comprises reacting the alkyl esters of 9-cis,11-trans conjugated linoleic acid or 10-trans,12-cis conjugated linoleic acid.

6. A method as claimed in claims 1 to 5, wherein the glyceride used is a synthetic or naturally occurring glyceridic oil or glyceridic fat or a derivative thereof.

7. A method as claimed in claim 6, wherein the glyceride used is sunflower oil, rapeseed oil, fish oil, soya oil, palm oil, safflower oil, linseed oil, wheatgerm oil, peanut oil, cottonseed oil, corn oil, milk fat, tung oil or shea butter or a derivative thereof.

8. A method as claimed in claims 1 to 7, which comprises using a positionally nonspecific lipase.

9. A method as claimed in claims 1 to 8, which comprises using a lipase specific for cis-Δ9-fatty acids or trans-Δ10-fatty acids.

10. A method as claimed in claims 1 to 9, wherein the lipase originates from microorganisms of the genera Burkholderia, Pseudomonas, Candida, Geotrichum, Chromobacterium, Corynebacterium, Staphylococcus or Aspergillus.

11. A method as claimed in claims 1 to 10, which comprises carrying out the reaction in the presence of water.

12. A method as claimed in claim 11, wherein the amount by weight of water is not greater than the amount by weight of glycerol or glyceride used.

13. A method as claimed in any of claims 1 to 12, wherein the alkyl alcohol is removed from the reaction mixture by distillation with reduced pressure.
